(19) United States
(12) Patent Application Publication
(10) Pub. No.: US 2007/0015701 A1
(43) Pub. Date: Jan. 18, 2007

(54) Macromolecular conjugates of bone morphogenetic protein-7

(76) Inventors: Samuel Zalipsky, Redwood City, CA (US); Francis X. Farrell, Doylestown, PA (US); Beth Hill, Sunnyvale, CA (US); Radwan Kiwan, Albany, CA (US)

Correspondence Address: PERKINS COIE LLP, P.O. BOX 2168, MENLO PARK, CA, 94026, US

(21) Appl. No.: 11/444,598

(22) Filed: May 31, 2006

Related U.S. Application Data
(60) Provisional application No. 60/686,505, filed on Jun. 1, 2005.

(51) Int. Cl. A61K 38/18

(52) U.S. Cl. 514/12; 424/78.27

(57) ABSTRACT

A modified bone morphogenetic protein (BMP, also referred to as bone morphogenic protein) composition is described. The bone morphogenetic protein, in one embodiment, is BMP-7 which is chemically modified with a hydrophilic polymer, such as poly(ethylene glycol).

### CROSS-REFERENCE TO RELATED APPLICATIONS

This application claims the benefit of U.S. Provisional Application No. 60/686,505, filed Jun. 1, 2005, incorporated herein by reference in its entirety.

### TECHNICAL FIELD

The subject matter described herein relates to a modification of bone morphogenetic proteins. More specifically, subject relates to a modified bone morphogenetic protein modified with one or more hydrophilic polymer chains.

### BACKGROUND

Cell differentiation is a central characteristic of morphogenesis which initiates in the embryo and continues to various degrees throughout the life of an organism in adult tissue repair and regeneration mechanisms. Proteins in the transforming growth factor-beta (TGF-beta) superfamily include subfamilies of highly-related genes that are involved in cell differentiation during development and/or during adult life. One of these subfamilies is the bone morphogenetic proteins, also referred to as bone morphogenic proteins or osteogenic proteins. These proteins were initially isolated from bone matrix and have bone-inducing activity. Subsequent isolation of the genes encoding these proteins from human cDNA libraries identified a family of protein, including BMP-2 through BMP-6 and osteogenic protein-1 (OP-1) which is also referred to as BMP-7 (Sampath T. K. et al., J. Biol. Chem., 267(28):20352 (1992)).

Isolated bone morphogenetic protein 7 (BMP-7) has been shown to induce cartilage and/or bone formation when administered to humans. It is also believed to assist in wound healing and tissue repair. More recently, BMP-7 has been proposed for use in treating diseased or damaged kidneys. Exogenously supplied BMP-7 protein tends to increase the rate of renal filtration, and in diseased kidney it tends to decrease in the rate of deterioration of the kidney function (Morrissey, J. et al., J. Am Soc Nephrol., 13:S4-S21 (2002); Simon, M. Am. J. Physiol Renal Physiol., F382-F389 (1 999); Zeisburg, M. et al., J. Biol Chem., 280(9):8094 (2005); Zeisburg, M. et al., Nat. Med., 9(7):964 (2003)). Treatment with the mature protein has also been shown to reduce renal fibrogenesis associated with ischemia and/or reperfusion injuries, as in ureteral obstruction (Hruska, K. A. et al., Am. J. Physiol Renal Physiol., 280:F130 (2000)).

However, delivery of BMP-7 as a therapeutic protein suffers from a number of problems. For example, the propensity of the protein to form bone at the site of injection is problematic for therapies when bone formation is not desired, as in wound healing, tissue repair, and treatment of kidney dysfunction. Another difficulty is its low solubility at neutral pH. While BMP-7 is soluble at pH<6.0, injections of acidic solutions are more painful and irritating to the patient than are solutions that are essentially at physiological pH. BMP-7 also suffers from the problem common to many proteins when administered via injection of a relatively fast in vivo clearance rate (T_{1/2}≈1.5 h in rats).

Thus, a modified BMP protein, and in particular, a modified BMP-7 protein, that addresses these difficulties in using BMP proteins as therapeutic agents is desired.

The foregoing examples of the related art and limitations related therewith are intended to be illustrative and not exclusive. Other limitations of the related art will become apparent to those of skill in the art upon a reading of the specification and a study of the drawings.

### BRIEF SUMMARY

In one aspect, a composition comprised of an isolated bone morphogenetic protein covalently attached to a hydrophilic polymer is provided.

In one embodiment, the bone morphogenetic protein is bone morphogenetic protein-7. In another embodiment, the bone morphogenetic protein-7 is human recombinant bone morphogenetic protein-7. An exemplary amino acid sequence of human bone morphogenetic protein-7 is given herein as SEQ ID NO:1.

In another embodiment, the hydrophilic polymer attached to the protein is poly(ethylene glycol). The poly(ethylene glycol) can be of any molecular weight, and in one embodiment has a molecular weight of between about 10-50 kDaltons.

In yet another embodiment, the bone morphogenetic protein is conjugated via two or more amino acid residues to said hydrophilic polymer.

In another aspect, a pharmaceutical preparation comprised of the composition described above and a pharmaceutically-acceptable vehicle is described.

In another aspect, a method of treatment is described, wherein the pharmaceutical preparation or the composition described above is administered via injection to a subject.

In another aspect, a composition of conjugates comprised of bone morphogenetic protein-7 modified with a hydrophilic polymer is described, the composition of conjugates prepared according to the process of reacting bone morphogenetic protein-7 with a functionalized hydrophilic polymer in the presence of an activating agent. Exemplary activation agents include of N-hydroxysuccinimide (HOSu), 1-hydroxybenzotriazole (HOBt), and hydroxyl-7-azabenzotriazole (HOAt).

In one embodiment, the functionalized hydrophilic polymer is an activated derivative of poly(ethylene glycol). An exemplary activated derivative of poly(ethylene glycol) is nitrophenyl carbonate derivatized methoxy-polyethylene glycol.

The composition formed by the process described herein, in one embodiment, provides a heterogenous composition of conjugates having a ratio of PEG:bone morphogenetic protein-7 of 1:1, 2:1 and 3:1. In one embodiment, the composition comprises more than about 50% of conjugates having a 1:1 ratio of PEG: bone morphogenetic protein-7.

In addition to the exemplary aspects and embodiments described above, further aspects and embodiments will become apparent by reference to the drawings and by study of the following descriptions.

### BRIEF DESCRIPTION OF THE DRAWINGS

FIG. 1 shows a synthetic reaction scheme for preparation of a poly(ethylene glycol)-BMP-7 conjugate referred to herein as “PEG30k-BMP-7”, where BMP-7 is covalently attached to nitrophenyl carbonate derivatized methoxy-polyethylene glycol (mPEG-NPC) in presence of N-hydroxysuccinimide (HOSu);

FIGS. 2A-2B are HPLC-SEC traces of the PEG30k-BMP-7 conjugate prior to purification, prepared according to the reaction shown in FIG. 1 (FIG. 2A) and of mature BMP-7 (FIG. 2B);

FIG. 3 shows the HPLC-SEC trace of the purified PEG30k-BMP-7 conjugate prepared according to the reaction shown in FIG. 1;

FIGS. 4A-4B are SDS-PAGE gels of the purified PEG30k-BMP-7 conjugate prepared according to the reaction shown in FIG. 1 stained with Coomassie blue for protein detection (FIG. 4A) and iodine for PEG detection (FIG. 4B). Lanes: 1, mature BMP-7; 2, PEG30k-BMP-7 conjugate; 3, fraction containing pure 1:1 PEG30k-BMP-7; 4, protein molecular weight standards on the left gel and PEG standards on the right; 5, 6, and 7 contain the same samples as in 1, 2, and 3 after thiolysis;

FIG. 5 shows another synthetic reaction scheme for preparation of a poly(ethylene glycol)-BMP-7 conjugate referred to herein as “PEG20k-Hz-BMP-7”, where the glyco portion of BMP-7 is oxidized and then reacted with mPEG-hydrazide;

FIGS. 6A-6C are HPLC-SEC traces for the mPEG20k-Hz-BMP-7 conjugate (FIG. 6A), the oxidized BMP-7 (FIG. 6B), and mature BMP-7 (FIG. 6C);

FIG. 7 shows the HPLC-SEC trace of the purified PEG20k-Hz-BMP-7 conjugate prepared according to the reaction shown in FIG. 5;

FIG. 8 is a plot showing induction of alkaline phosphatase in rat osteoblastic (ROS) cells in vitro upon exposure to various concentrations, in ng/mL, of the PEG30k-BMP-7 conjugate;

FIGS. 9A-9C are plots of interleukin-6 (IL-6) concentration, in pg/mL, as a function of BMP-7 concentration, in ng/mL, when human kidney cells in vitro are exposed to mature BMP-7 (squares), PEG20k-Hz-BMP-7 (inverted triangles) or PEG30k-BMP-7 (triangles), the concentration of BMP-7 ranging from 50 ng/mL to 400 ng/mL;

FIGS. 10A-10C are plots of interleukin-8 (IL-8) concentration, in pg/mL, as a function of BMP-7 concentration, in ng/mL, when human kidney cells in vitro are exposed to mature BMP-7 (squares, control), PEG20k-Hz-BMP-7 (inverted triangles) or PEG30k-BMP-7 (triangles), the concentration of BMP-7 ranging from 50 ng/mL to 400 ng/mL;

FIG. 11 is a graph of BMP-7 serum concentration, in ng/mL, as a function of time, in hours, after intravenous injection of mature BMP-7 (triangles), PEG30k-BMP-7 (inverted triangles), or saline vehicle (squares); and

FIGS. 12A-12B are bar graphs showing the control normalized level of alpha-smooth muscle actin (FIG. 12A) and collagen alpha1(l) (FIG. 12B) in mice after unilateral ureteral obstruction and treatment with mature BMP-7 (dotted bars), soluble BMP-7 (cross-hatched bars), PEG30k-BMP-7 (dotted bars).

### DETAILED DESCRIPTION

I. Definitions

“BMP-7” refers to bone morphogenetic protein-7. Amino acid sequences of BMP-7 for a variety of species are known, including but not limited to, Homo sapiens (GenBank Accession No. MG43508; NP_{—}001710), Gallus gallus (chicken, GenBank Accession No. AAF34758), Canis familiaris (dog, GenBank Accession No. P34819 and AAF89752), Sus scrofa (pig, GenBank Accession No. AAV381 11), Oryctolagus cuniculus (rabbit, GenBank Accession No. AAL24500), Rattus norvegicus (rat, GenBank Accession No. XP_{—}342592), Mus musculus (house mouse, GenBank Accession Nos. P23359 and NP_{—}031583), and Ovaris aries (sheep, GenBank Accession No. MM46923). Thus, the term “BMP-7” refers to an amino acid sequence of a BMP-7 from any species, and to fragments and variations of these proteins that retain the desired therapeutic activity of the mature protein. Those of skill in the art recognize that certain portions of a protein are required for therapeutic activity, permitting omission of selected amino acid residues and/or substitution of selected amino acid residues. It will also be appreciated that individual amino acid residues in the protein may be modified by oxidation, reduction, or other derivatization, and the protein may be cleaved to obtain fragments which retain activity. Such alterations which do not destroy biological activity do not remove the protein sequence from the definition. More generally, the term BMP-7 intends any protein sequence that has at least about 70%, more preferably 75%, still more preferably 80%, still more preferably 85%, still more preferably 90%, and still more preferably 95% sequence identity to at least one of the GenBank sequences noted above or to other sequences noted below. Sequence “identity” is determined by comparing the amino acid sequences of polypeptides when aligned so as to maximize overlap and identity while minimizing sequence gaps. The percent identity of two amino acid or two nucleic acid sequences can be determined by visual inspection and/or mathematical calculation, or more preferably, the comparison is done by comparing sequence information using a computer program. An exemplary, preferred computer program is the Genetics Computer Group (GCG; Madison, Wis.) Wisconsin package version 10.0 program, ‘GAP’ (Devereux et al., Nucl. Acids Res., 12: 387 (1984)). Other programs used by those skilled in the art of sequence comparison can also be used, such as, for example, the BLAST (BLASTP) and BLASTN programs, available for use via the National Library of Medicine website http://www.ncbi.nlm.nih.gov/BLAST. In preferred embodiments, sequences are considered homologous or identical to one another if their amino acid sequences are at least about 60% identical, more preferably at least 70% identical, still more preferably at least 80% identical, when determined from a visual inspection or from one of the aforementioned computer programs.

“Hydrophilic polymer” as used herein refers to a polymer having moieties soluble in water, which lend to the polymer some degree of water solubility at room temperature. Exemplary hydrophilic polymers include polyvinylpyrrolidone, polyvinylmethylether, polymethyloxazoline, polyethyloxazoline, polyhydroxypropylyoxazoline, polyhydroxypropyl-methacrylamide, polymethacrylamide, polydimethyl-acrylamide, polyhydroxypropylmethacrylate, polyhydroxyethylacrylate, hydroxymethylcellulose, hydroxyethylcellulose, polyethyleneglycol, polyaspartamide, copolymers of the above-recited polymers, and polyethyleneoxide-polypropylene oxide copolymers. Properties and reactions with many of these polymers are described in U.S. Pat. Nos. 5,395,619 and 5,631,018.

“PEGylation” refers to the attachment of one or more polyethylene glycol (PEG) substituents or derivatives to a bone morphogenetic protein, such as BMP-7. In a preferred embodiment, the term refers to covalent attachment of one or more PEG substituents or derivatives by a bond that is not reversible or labile under physiological conditions. Thus, a “covalently” attached polymer refers to a linkage that is not generally considered to be reversible or labile under physiological conditions.

II. Composition

In one aspect, a composition comprised of BMP-7 modified by covalent attachment to a hydrophilic polymer is provided. In a preferred embodiment, BMP-7 is human BMP-7, which can be recombinantly produced in Chinese hamster ovary (CHO) cells. As noted above, human BMP-7 induces bone formation in vivo and increases cell proliferation and collagen synthesis of osteoblasts in vitro (Sampath T. K. et al., J. Biol. Chem., 267(28):20352 (1992)). Recombinant human BMP-7 consists of a 34-38 kDa disulfide linked homodimers identified herein as SEQ ID NO:1. Upon reduction, the homodimers migrate as 23, 19, or 27 kDa monomers containing amino acid residues 293-431 of SEQ ID NO:1, where the amino terminal Met residue of the pre-pro BMP-7 is defined as residue 1 (Ozkaynak, E., EMBO J., 9:2085 (1990)). Residues 29-292 (SEQ ID NO:2) are termed the pro-domain (Jones, W. K. et al., Growth Factors, 11:215 (1994)). Digestion of the monomers with N-glycanase reduces the 23, 19, and 17 kDa monomers to a single 14 kDa species indicating that the apparent molecular weight differences are due to differential glycosylation (Jones, Id.). The purified BMP-7 homodimers are sparingly soluble in physiological buffers, such as phosphate buffered saline or cell culture media, and require denaturants to remain in solution. The mature domain of recombinant BMP-7 corresponds to residues 293-431 of SEQ ID NO:1 and is referred to herein as “mature” BMP-7 and is identified herein as SEQ ID NO:3.

In a preferred embodiment, human BMP-7 having a sequence identified herein as SEQ ID NO:3 is used in the composition. In one embodiment, the composition includes a human BMP-7 having at least about 70% sequence identity to SEQ ID NO:3, where sequence identity is determined as described above. In other embodiments, the composition includes a human BMP-7 having at least 80%, preferably 85%, more preferably 90%, still more preferably 95% sequence identity to the protein sequence identified as SEQ ID NO:3.

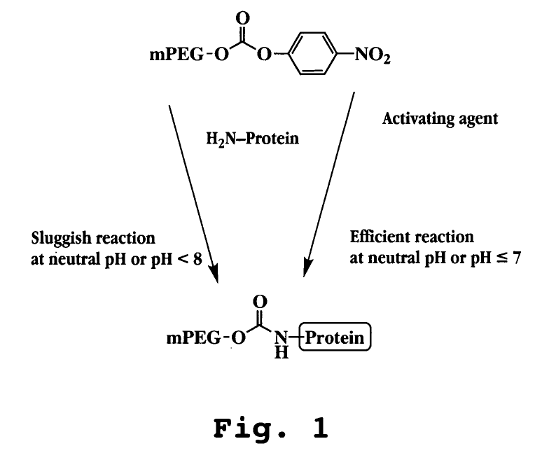

BMP-7 is chemically modified with one or more hydrophilic polymer chains. Chemical attachment, or conjugation, of hydrophilic polymers to proteins is exemplified in the art with the hydrophilic polymer poly(ethylene glycol) (PEG), however it will be appreciated that other hydrophilic polymers, such as those listed above, are equally suitable. Conjugation of a PEG chain to a protein, such as BMP-7, is typically one using an “activated derivative” of PEG, i.e, a PEG having a functional group at one or more terminal ends for reaction with, for example an amino group on the protein. Many activated derivatives of PEG are known in the art, including the pyridyldithiopropionyl-PEG, tert-butyloxycarbonyl-HN-PEG, succinimidyl carbonate-PEG, nitrophenyl carbonate-PEG, and others set forth in Zalispky, S. et al., Methods in Enzymology, 387:50 (2004). Functional moieties on PEG that attach to amino groups of the protein, or a glycan portion of the protein, or the N-terminus of the protein are suitable, with acylation of the amino groups a common method. Conjugates of BMP-7 and PEG were prepared in supporting studies by two exemplary reaction schemes, detailed in Examples 1 and 2. With respect to Example 1, human mature BMP-7 (SEQ ID NO:3) was mixed with nitrophenyl carbonate derivatized methoxy-PEG (mPEG-NPC) in the presence of an activating agent, exemplified by N-hydroxysuccinimide (HOSu). As seen in FIG. 1, which shows an amine-directed PEGylation of BMP-7, reaction of BMP-7 with the acylating agent mPEG-NPC at a neutral pH in the range of 6-7.5 is proceeds slowly. PEGylation of proteins with PEG-NPC proceeds more quickly at a pH greater than 7.5, as PEG-NPC is more reactive under basic conditions. As noted above, BMP-7 is sparingly soluble in water at neutral and basic pH, making it difficult to chemically modify the protein with efficiency using mPEG-NPC. However, in one embodiment, a conjugate of BMP-7 chemically modified with PEG is prepared by reacting BMP-7 with an acylating agent, like mPEG-NPC, in the presence of an activating agent, as also illustrated in FIG. 1. Addition of the activating agent to the reaction mixture increases the reaction efficiency and allows facile protein modification at neutral pH values (e.g. between 6-7.5) and at pH values below 7.0.

The activating agent in FIG. 1 and in Example 1 is exemplified by HOSu. However, it will be appreciated that any water-soluble, non-carboxylic, Brönstead acid of moderate acidity having the propensity to donate N- or O-linked protons to the PEGylation reagent is suitable for use as the activating agent. General examples include acidic alcohols, phenols, imidazols, triazols and tetrazols, among others. Examples of acidic acids suitable for use include, but are not limited to, N-hydroxydicarboxyimides, N-hydroxyphthalimides particularly with nitro and other electron withdrawing substituents on the aromatic ring, N-hydroxy tetrahydrophthalimide, N-hydroxyglutarimide, N-hydroxy-5-norbornene-2,3-dicarboxyimide, and N-hydroxy-7-oxabicyclo[2.21]hept-5-ene-2,3-dicarboxyimide. 1-N-hydroxybenzotriazol and derivatives with electron withdrawing groups on the aromatic ring, e.g. nitro, chloro, 3-hydroxy-1,2,3-benzotriazin-4(3H)-one. N-hydroxysulfosuccinimide sodium salt is very soluble in water, which means that it can be used at even higher concentration in aqueous buffers than HOSu. Exemplary hydroxy amines include, in addition to HOSu, sulfonate derivatives of HOSu, 1-hydroxybenzotriazole (HOBt), and hydroxyl-7-azabenzotriazole (HOAt). These compounds act as an efficient buffer component in a pH range of about 4 to about 7.5, based on their pKa. For example, HOSU, being a weak acid of pKa=6, acts as an efficient buffer component in a pH range of about 5 to about 7. The activating agent may be added to a buffer, or may comprise the buffer with or without other salts. Further, as HOSu is very soluble in an aqueous solution, it can be added to buffers at relatively high concentration to further boost the PEGylation reaction.

Other compounds suitable as the activating agent include, phenols , such as dinitrophenol, trinitrophenol, trifluorophenol, pentafluorophenol, and pentachlorophenol, 4- or 2-hydroxypyridine and derivatives are also suitable, e.g. hydroxyl-2-nitropyridine, as well as compounds having an acidic N—H functionality, such as imidazol derivatives with electron withdrawing groups (imidazol, pKa=7), e.g. 4- or 2-nitroimidazol, triazol, tetrazol, and some derivatives, such as 2-nitro-1,2,4-triazole.

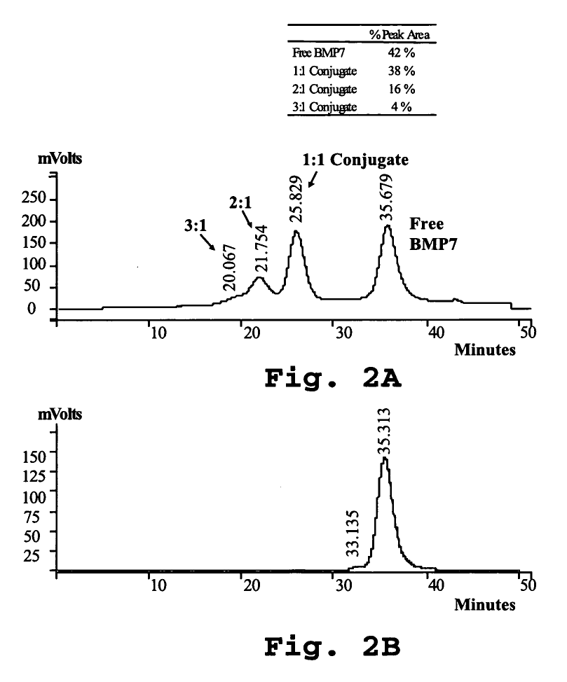

As described in Example 1, a PEG-modified BMP-7 was prepared by reacting the mature protein with a functionalized PEG, mPEG-NPC, (mPEG molecular weight of 30 kD), in the presence of HOSu as the activating agent. The resulting PEG-BMP-7 conjugate, referred to herein as mPEG30 kDa-BMP-7, was characterized by HPLC size exclusion chromatography prior to purification. As seen in FIG. 2A, about 58% of the BMP-7 was PEGylated, with formation of mono- and di-PEGylated-BMP-7 being favored. Mature BMP-7, which elutes at about 35 minutes (FIG. 2B), was 42% of the total peak area. The PEG30k-BMP-7 composition was comprised of 1:1 PEG-BMP-7 conjugates (38%), 2:1 PEG-BMP-7 conjugates (16%), and 3:1 PEG-BMP-7 conjugates (4%).

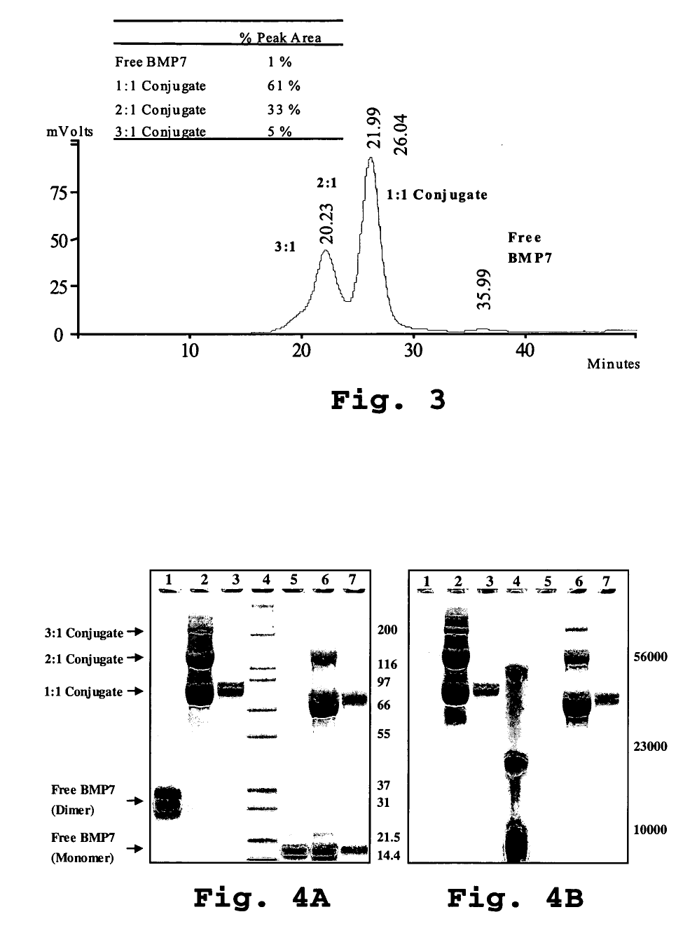

The composition was purified to remove unreacted protein using dialysis and ion exchange chromatography and characterized again by HPLC-SEC and gel electrophoresis. FIG. 3 shows the HPLC-SEC trace PEG30k-BMP-7 after purification. After purification, 61% of the conjugate composition was 1:1 PEG-BMP-7. 2:1 PEG-BMP-7 conjugate and 3:1 PEG-BMP-7 conjugate comprised 33% and 5% of the composition.

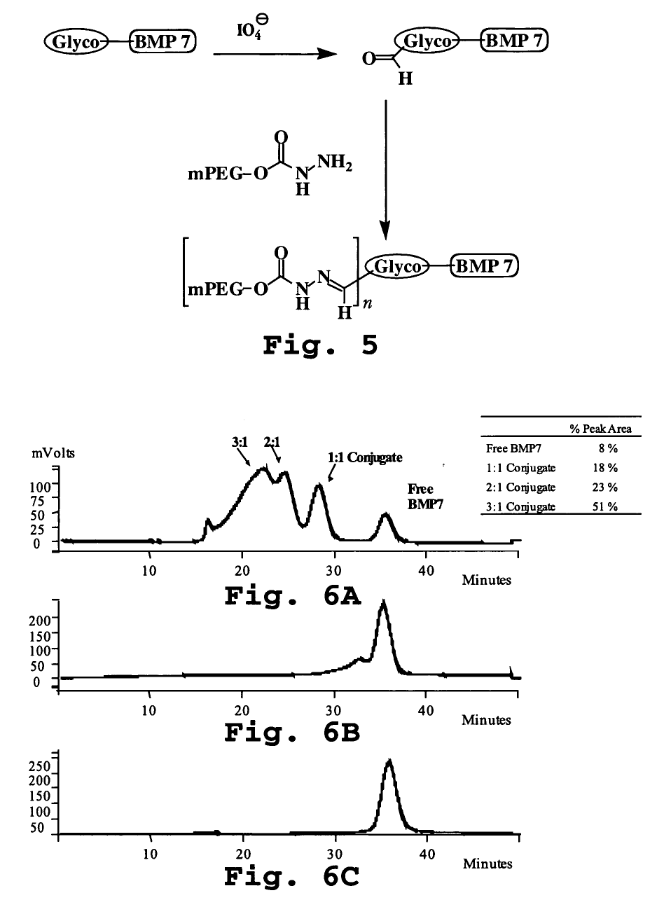

FIGS. 4A-4B are photographs of the electrophoresis gels stained with Coomassie blue for detection of protein (FIG. 4A) and with iodine for detection of the PEG (FIG. 4B). In both gels, Lane 1 is mature BMP-7, Lane 2 is the purified preparation of PEG30k-BMP7, Lane 3 is the PEG30k-BMP7 1:1 conjugate. Lane 4 is a molecular weight (MW) marker for proteins in panel A and a molecular weight marker for PEG in panel B. Lanes 5, 6, and 7 correspond to the proteins of Lanes 1, 2, and 3, respectively, after reduction with mercaptoethanol. The gels confirm the results from the HPLC-SEC, showing that formation of the 1:1 PEG-protein conjugate was favored.

Another study was conducted where a modified BMP-7 was prepared using a carbohydrate-directed coupling reaction, as described in Example 2 and illustrated in FIG. 5. In this approach, BMP-7 was oxidized with sodium periodate and then reacted with mPEG-hydrazide (mPEG-Hz), the PEG having a molecular weight of 20 kDa. The resulting composition prior to purification was characterized using HPLC-SEC, as shown in FIGS. 6A-6C. The trace for mature BMP-7 is shown in FIG. 6C, the protein elutes at about 36 minutes. The oxidized protein is shown in FIG. 6B, and elutes at about 35 minutes. After reaction with mPEG-Hz, a small amount (8%) of mature protein remained, with the remaining 92% modified with one or more PEG chains. The majority of the conjugates were 3:1 PEG-protein (51%), with the 2:1 and 1:1 PEG-protein conjugates comprising 23% and 18% of the total.

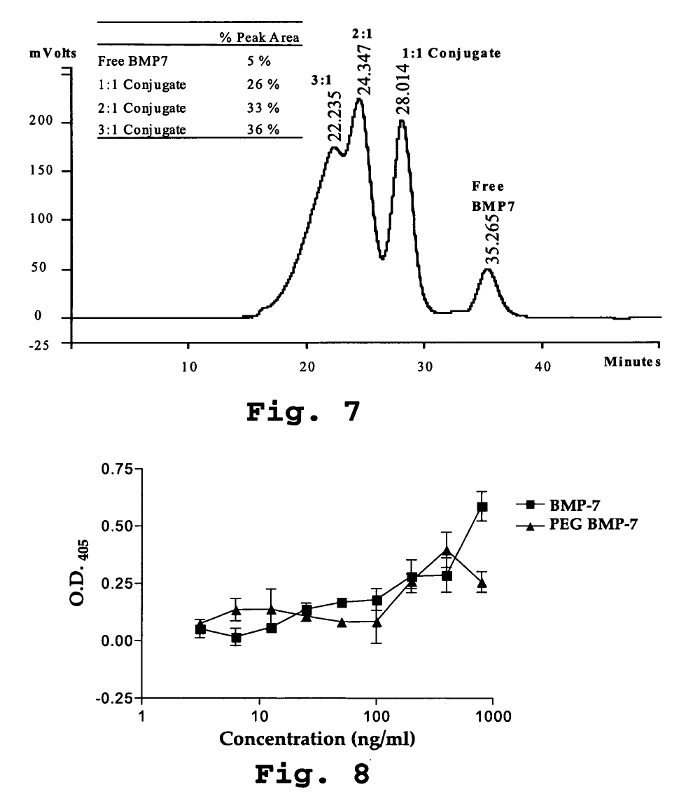

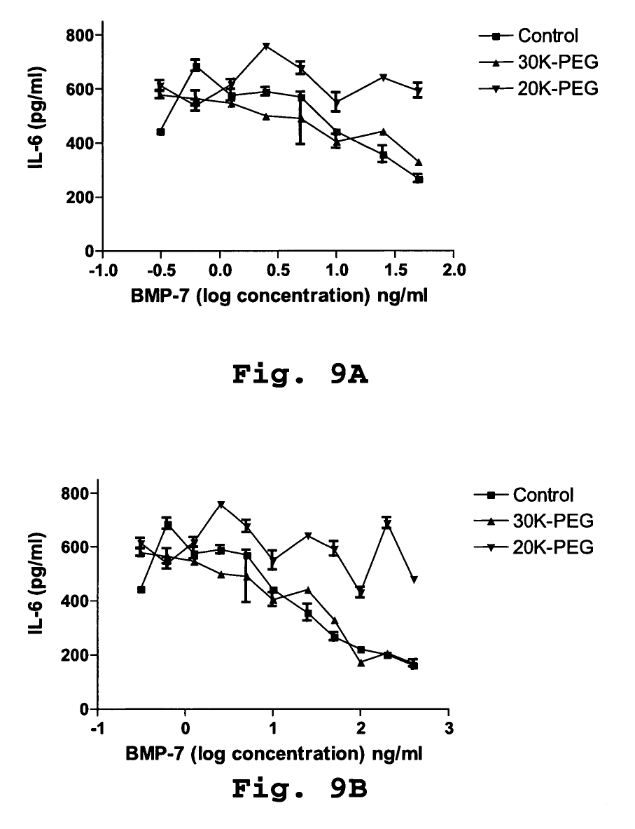

The PEG20k-Hz-BMP-7 composition was purified by dialysis and ion exchange chromatography and analyzed again by HPLC-SEC. The trace of the composition after purification is shown in FIG. 7. The purified preparation contained 95% of PEGylated BMP-7 and approximately 5% of free protein, with the PEGylated protein fractions as indicated in the figure.

The PEG-modified BMP-7 exhibited improved water solubility properties compared to the mature protein. Mature BMP-7 is water soluble at acidic pH (pH≦5.0), however upon neutralization the protein precipitates. In contrast, PEG-BMP-7 remains soluble in water above pH 7 and does not precipitate upon neutralization with common buffer salts.

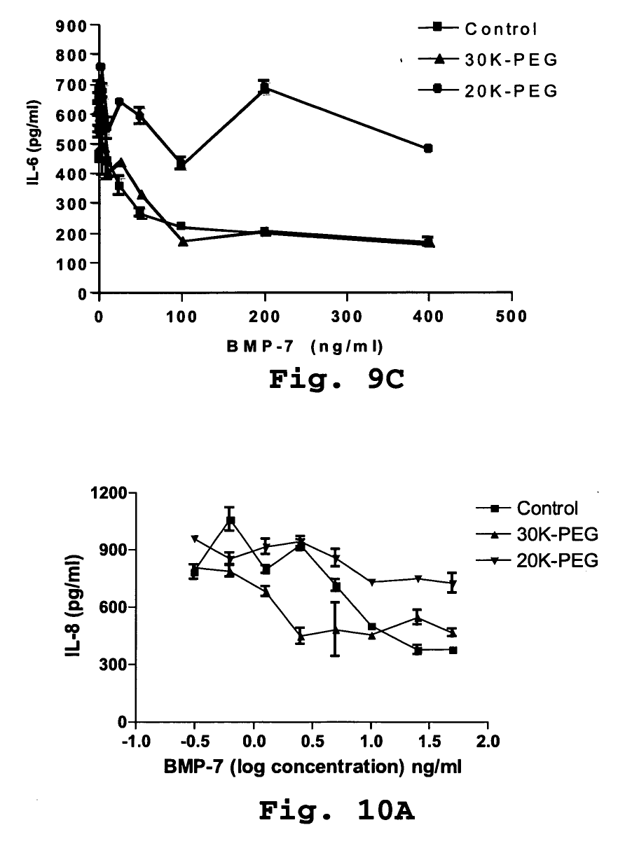

The modified BMP-7, PEG30 kDa-BMP-7, was tested in vitro to determine if the conjugate retains biological activity. As described in Example 3, the ability of the conjugate to induce alkaline phosphatase specific activity in rat osteoblastic (ROS) cells was evaluated. ROS cells were incubated with various concentrations of mature BMP-7 and PEG30 kDa-BMP-7 for 40-56 hours. Induction of alkaline phosphatase was assessed by lysing the cells and analyzing for the presence of alkaline phosphatase via absorbance. The results are shown in FIG. 8. FIG. 8 shows the absorbance (O.D. 405 nm) as a function of BMP-7 concentration, in ng/mL, of mature BMP-7 (squares) and of the PEG30k-BMP-7 conjugate (triangles). The PEG30K-BMP-7 conjugate exhibited similar activity to the mature BMP-7. This result was not expected since proteins, and particularly receptor-binding proteins, lose a substantial portion of their in vitro biological activity upon PEGylation.

Biological activity of the PEG-modified BMP-7 conjugate was also tested in vitro using a human kidney (HK-2) cell line. Mature (unpegylated) BMP-7 inhibits production of the cytokines interleukin-6 (IL-6) and interleukin-8 (IL-8). The conjugates prepared as described in Examples 1 and 2, PEG30k-BMP-7 and PEG-20k-Hz-BMP-7, were tested according to the procedures described in Example 4 to determine whether, after chemical modification with PEG, the protein retained its ability to inhibit IL-6 and IL-8 production in HK-2 cells.

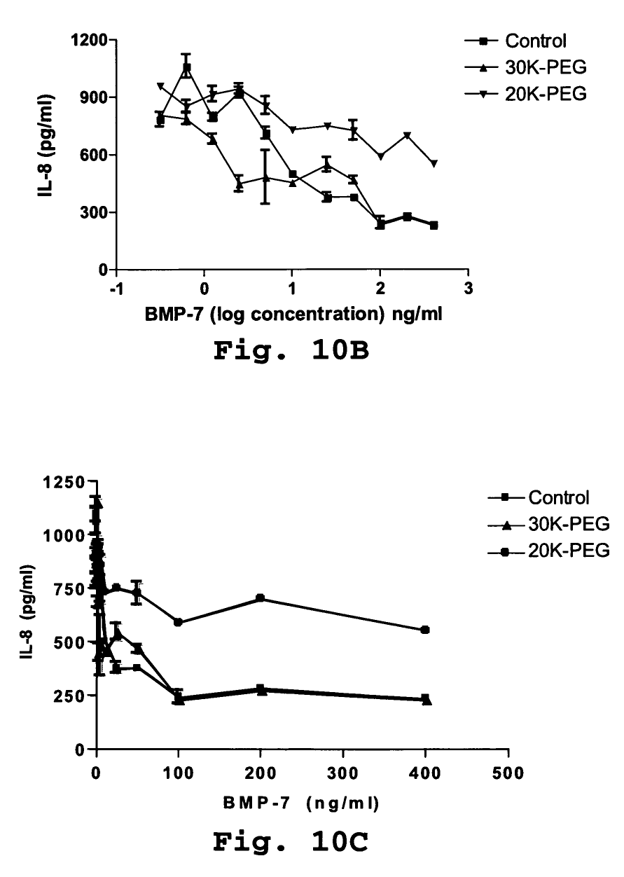

FIGS. 9A-9C show that the PEG-BMP-7 conjugates retain biological activity, as evidenced by the ability to inhibit IL-6 production. FIG. 9A shows that the conjugated proteins inhibit IL-6 production over a protein concentration range of 50 ng/mL to 1.75 ng/mL, with the mature, mature BMP-7 (control, squares) and PEG30k-BMP-7 (triangles) having essentially the same inhibitory activity. FIGS. 9B and 9C are similar plots over a wider protein concentration range and show that the PEG30k-BMP-7 conjugate retains activity nearly identical to that of the mature, mature protein, with the PEG-20k-Hz-BMP-7 conjugate have a somewhat diminished activity at higher protein concentrations.

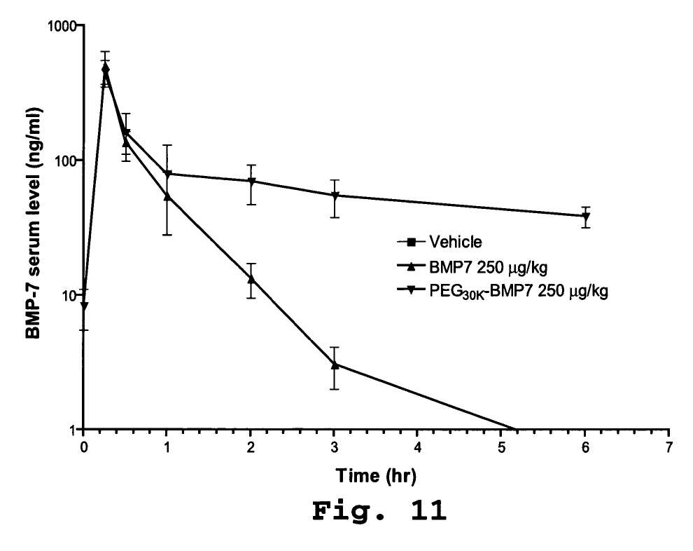

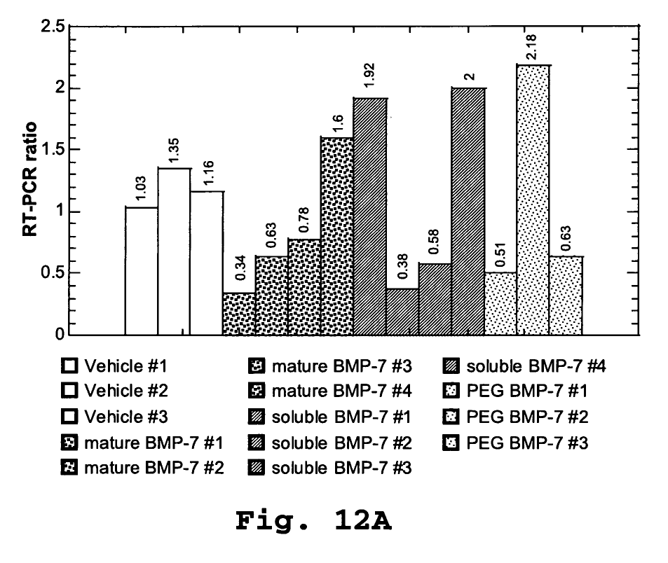

FIGS. 10A-10C show the results for interleukin-8 (IL-8), where mature BMP-7 (squares) and the PEG-BMP-7 conjugates (triangles) inhibit production of IL-8 at low concentrations (FIG. 10A). At higher concentrations of protein (FIGS. 10B, 10C), the PEG-20k-Hz-BMP-7 conjugate have a somewhat diminished activity (inverted triangles) relative to mature BMP-7, yet still exhibited an ability to inhibit IL-8 production. The PEG30k-BMP-7 conjugate (triangles) retained IL-8 inhibitory activity essentially equivalent to the mature protein.

As noted above, a problem associated with systemic administration of mature BMP-7 for the treatment of renal failure and osteodystrophy is bone formation at the site of injection. Three studies were conducted in vivo to evaluate bone formation at the site injection after delivery of the conjugate PEG30k-BMP-7. These studies are described in Example 5-7. In the study detailed in Example 5, rats were treated with mature BMP-7 or with the PEGylated protein by subcutaneous or by intramuscular injection. Approximately two weeks after injection, the injection site was analyzed by X-ray. Tissue from the right flank of each animal was harvested and assessed for visible signs of bone nodules. The tissue was also processed for quantitative 3D microCT analysis. Bone nodules formed in the muscle tissues of about 95% of the animals that received an intramuscular injection of mature BMP-7. In contrast, no bone was observed at the intramuscular injection sites of the animals treated with the PEG-BMP-7 conjugate.

The studies described in Example 6 and 7 provide further confirmation of the results observed in the study of Example 5. Pegylation of BMP-7 prevented injection site ossification in the muscle and skin, indicating that the conjugate is less osteogenic, or more soluble, and capable of rapid dispersion after injection. The results of the studies described in Examples 5 and 6 are summarized in Table 1.

TABLE 1

| | | | Route | Bone | |
|---|---|---|---|---|---|
| | | | of | formation | MicroCT |
| | Number | | Adminis- | (X ray | (mm³, |
| Group | of rats | Sample | tration | analysis) | average) |
| 1 | 5 | BMP7 | I.M. | 4/5 | 7.23 |
| (Example 5) | | | | | |
| 2 | 5 | PEG-BMP7 | I.M. | 0/5 | 0 |
| (Example 5) | | | | | |
| 3 | 5 | BMP7 | S.C. | 0/5 | 1.83 |
| (Example 5) | | | | | |
| 4 | 5 | PEG-BMP7 | S.C. | 0/5 | 0 |
| (Example 5) | | | | | |
| 1 | 5 | BMP7 | I.M. | 5/5 | 9.88 |
| (Example 6) | | | | | |
| 2 | 5 | PEG-BMP7 | I.M. | 0/5 | 0 |
| (Example 6) | | | | | |

The pharmacokinetics of PEGylated-BMP-7 were evaluated by injecting the protein intravenously into rats, as described in Example 8. The blood circulation lifetimes of the conjugate and of mature BMP-7 are shown in FIG. 11. The increased blood circulation lifetime of the PEG30k-BMP-7 conjugate (inverted triangles) relative to mature BMP-7 (triangles) is apparent.

III. Methods of Use

In another aspect, treatment methods comprising administering a conjugate comprised of BMP-7 covalently attached to a hydrophilic polymer, such as poly(ethylene glycol) are provided. For example, BMP-7 has a beneficial effect on renal structure and function in patients suffering from acute or chronic renal injury. Administration of BMP-7 in the form of a PEGylated conjugate can increase glomerular filtration rate and renal blood flow, with a decrease in serum creatinine and blood urea nitrogen. BMP-7 also has a beneficial effect in preserving the integrity of vascular smooth muscle cells and in maintaining actin expression in alpha-smooth cells. Thus, the conjugate finds particular use in the prevention and treatment of renal conditions, exemplified by but not limited to, renal fibrosis, renal ischemic and/or reperfusion injury, and other conditions evident to those of skill in the art.

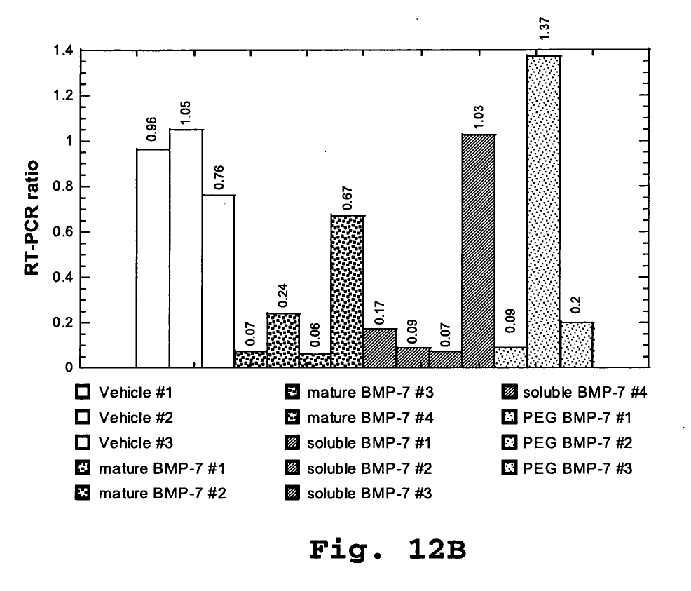

An exemplary treatment using the PEGylated BMP-7 conjugate is illustrated in Example 9. In this study, a mouse unilateral ureteral obstruction (UUO) model was used to evaluate efficacy of the PEGylated PMB-7 conjugate. The ureters of mice were ligated to block ureteral flow. The ability of PEG30k-BMP-7 to suppress progression of renal fibrosis during a four day period of ureteral obstruction was evaluated. Using real time reverse transcriptase polymerase chain reaction (RT-PCR) the relative level of the relative level of a-smooth muscle actin expression was determined. The level of expression in vehicle treated animals was set to one unit for convenient comparison. The results are shown in FIG. 12A. The relative level of a-smooth muscle actin in the four day obstructed kidney was reduced in three out of four mice treated with mature BMP-7, in two out of four mice treated with soluble BMP-7, and in two out of three mice treated with PEG BMP-7.

By real time RT-PCR the relative level of collagen α1(I) expression in the animals was also determined. The results are shown in FIG. 12B. The level of expression in vehicle treated animals was set to one unit. The relative level of collagen α1(I) in the four day obstructed kidney was reduced in three out of four mice treated with mature BMP-7, in three out of four mice treated with soluble BMP-7, and in two out of three mice treated with PEG BMP-7.

For use in such methods of treatment, the conjugate composition described above can be formulated for parenteral delivery according to methods well known in the art. Typically, the protein is formulated with a suitable carrier vehicle, such as saline or other vehicle that is pharmaceutically-approved.

It will also be appreciated that the PEG-BMP-7 conjugate can be administered in combination with other therapeutic agents. A specific example includes co-treatment of PEG-BMP-7 with enalapril.

### IV. EXAMPLES

The following examples further illustrate the subject matter described herein and are in no way intended to limit its scope.

### Example 1

### Preparation of Poly(ethylene glycol)-BMP-7 Coniugate (mPEG30k-BMP-7)

This reaction scheme is shown in FIG. 1.

A. Coniugation Reaction and Purification

Recombinant human bone morphogenetic protein-7 (BMP-7; SEQ ID NO:3) was obtained as a lyophilized powder and kept at −70° C. A 1.4 mg/mL BMP7 stock solution was prepared in 25 mM N-hydroxysuccinimide (HOSu), pH 6.

Nitrophenyl carbonate derivatized methoxy-polyethylene glycol, molecular weight of 30,000 Daltons (mPEG30k-NPC), was purchased from NOF Corporation (Tokyo, Japan). A 10 mM stock solution of mPEG30k-NPC was prepared in acetonitrile.

The reaction (FIG. 1) was initiated by mixing 12.86 mL of BMP-7 (18 mg) to 4.24 mL of HOSu buffer, pH 6. Afterward, 0.9 mL of mPEG30k-NPC were added drop by drop to the mixture, while gently vortexing. The reaction was allowed to proceed for 16 hours at room temperature (21-22° C.) on a rocking mixer. The final reaction volume was 18 mL containing 1 mg/mL (0.028 mM) of BMP-7, 0.5 mM of mPEG30k-NPC, 5% acetonitrile, and a molar ratio of 18 PEG/BMP-7. The final HOSu concentration was 24 mM, which is approximately 48 molar excess over mPEG30k-NPC. The reaction was quenched with 10 mM glycine for 1 hour at room temperature.

The product from the conjugation reaction was analyzed by HPLC-SEC using Superose6 10/300 GL, 1×30 cm column (GE Healthcare, Piscataway, N.J.), and 25 mM Tris, 300 mM NaCl, 6 M Urea, pH 6.5, mobile phase. The sample was diluted 1/20 in the mobile phase, and 50 μL was injected to the column. The flow rate was set at 0.5 mL/min, and elution off the column was monitored by a fluorescence detector set at an excitation wavelength of 295 nm, and emission wavelength of 360 nm (bandwidth 15 nm). The results for the conjugate and for the BMP-7 reference are shown in FIGS. 2A-2B, respectively.

For purification, the conjugation reaction sample was dialyzed in 10 mM sodium acetate buffer pH 5, using SPECTRA/POR 1 membrane tubing (Spectrum Medical Industries Inc., Los Angeles, Calif.), having a molecular weight cut-off of 6000-8000. The dialysis was carried out at 4° C. At the end of dialysis, the sample was filtered through a 0.45 μm Acrodisc HT Tuffryn membrane syringe filter (PALL Life Sciences, Ann Arbor, Mich.).

Then, a sulphopropyl cation exchanger column, Source 15S PE 4.6×100 mm (GE Healthcare, Piscataway, N.J.), 1.7 mL total volume, was equilibrated with 20 column volumes of 10 mM sodium acetate pH 5 buffer. Next, 20 mL of the dialyzed conjugation sample were loaded on the column. Elution was performed by gradient elution using mobile phase A containing 10 mM sodium acetate pH 5, mobile phase B1 containing 1 M NaCl in 10 mM Na Acetate pH 5, and mobile phase B2 containing 6 M Urea, 1 M NaCl, 10 mM Na Acetate pH 5, at a flow rate of 1 ml/min. The unbound material to the column was washed out with 40 mL of mobile phase A. The gradient elution started by increasing mobile phase B1 from 10% to 60% in 50 minutes, then to 100% B2 (1 M NaCl, 6 M Urea) for 10 minutes at 2 mL/min. Fractions were collected at 1 mL/fraction throughout the elution step. Unreacted PEG did not bind to the column and came out with flow-through material. PEGylated BMP7 started eluting at approximately 150 mM NaCl and was completed at approximately 400 mM NaCl. Free BMP7 was eluted at the end with 1 M NaCl containing 6 M urea.

In order to identify the content of the fractions collected throughout the ion exchange separation, aliquots from the fractions were analyzed by HPLC-SEC using Superose6 10/300 GL column described above (results not shown). Fractions from the ion exchange separation containing the PEGylated protein were pooled, concentrated, and dialyzed in 20 mM sodium acetate, 5% mannitol, pH 4.5 buffer, under nitrogen at 20 psi, in a 10 mL Amicon ultrafiltration stirred cell (Millipore Corp., Billerica, Mass.), using an OMEGA ultrafiltration membrane disc filter (PALL Life Sciences, Ann Arbor, Mich.), having a molecular weight cut-off of 3000. The sample volume was brought down to approximately 3.5 mL final volume.

The concentrated sample was sterile filtered through 0.22 μm Acrodisc HT Tuffryn membrane syringe filter, and sterilely filled into autoclaved glass vials. All vials were stored at 4° C. Approximately a total of 1.2 mg of PEG30k-BMP-7 was obtained from the purification, as determined by a protein assay described below.

B. Protein Determination Assay

The protein determination assay was based on the fluorescent characteristic of the protein's intrinsic tryptophan. BMP-7 was used as a standard, and serial dilutions were made at 6.25, 12.5, 25, 50,100, and 200 μg/mL in 20 mM sodium acetate, 5% mannitol, pH 4.5 buffer. The mPEG30k-BMP-7 sample was diluted 1:10 and 1:20 in the same buffer. The standards and test samples were transferred to a black microtiter plate, at 200 μL/well, in triplicates. The plate was read in a fluorometer set at an excitation wavelength of 295 nm (2 nm slit), and emission wavelength of 360 nm (10 nm slit). The results are shown in Table A.

TABLE A

| | | Estimated | | | |
|---|---|---|---|---|---|
| | | BMP-7 | | Estimated | Protein |
| Test | Mean | concentration | | concentration × | concen- |
| sample | Intensity | in test sample | Dilution | dilution factor | tration |
| dilution | cps | μg/mL | factor | mg/mL | mg/mL |
| 1/20 | 25521 | 16.79 | 20 | 0.34 | 0.35 |
| 1/10 | 51879 | 35.45 | 10 | 0.35 | |

B. Characterization

B1. HPLC-SEC Analysis

The purified mPEG30k-BMP7 sample was analyzed by size exclusion chromatography using Superose6 10/300 GL column described above. The sample was diluted to 50 μg/mL in the mobile phase, and 50 μL were injected to the column. The flow rate was set to 0.5 mL/min, and elution off the column was monitored by a fluorescence detector set at an excitation wavelength of 295 nm, and emission wavelength of 360 nm (bandwidth 15 nm). The results are shown in FIG. 3 for the mature protein and for the conjugate.

B2. SDS-PAGE Analysis

The mPEG30k-BMP7 sample was analyzed by gel electrophoresis under denaturing conditions, using NuPAGE® Bis-Tris 4-12% gradient gel and MOPS-SDS running buffer (Invitrogen Life Technology, Carlsbad, Calif.). Samples and controls were loaded on 2 gels at 10 μL/well containing 1.5 to 5 μg of protein. The gels were run at a constant voltage of 200 volts for 55 minutes. One gel was stained in Coomassie Blue for protein detection and the other in iodine for PEG detection, as shown in FIGS. 4A-4B, respectively.

### Example 2

### Preparation of Poly(ethylene glycol)-BMP-7 Conjugate (mPEG20k-Hz-BMP-7)

This reaction scheme is shown in FIG. 5.

A. Coniugation Reaction

A two step reaction of oxidation and conjugation was performed using recombinant human BMP-7. A 2.8 mg/mL BMP7 stock solution was prepared in 25 mM sodium acetate pH 5 buffer (15.6 mg total BMP-7). The BMP-7 oxidation reaction was carried out in 1 mM sodium periodate for 20 minutes at 4° C., then quenched with 2 mM N-acetyl-methionine.

Then, a 5 mM stock solution of methoxy-polyethylene glycol 20000-hydrazide (mPEG20k-Hz) was prepared in 25 mM sodium acetate buffer pH 5. The oxidized BMP-7 (14 mg) was reacted with the mPEG20k-Hz at 1.4 mg/mL of BMP-7 (0.039 mM) and 2.9 mM mPEG20k-Hz, resulting in a molar ratio of 75/1 PEG per protein, for 16 hours at room temperature (21-22° C.) on a rocking mixer.

The product of the conjugation reaction was analyzed by HPLC-SEC using Superose6 10/300 GL, 1×30 cm column (GE Healthcare, Piscataway, N.J.), and 25 mM Tris, 300 mM NaCl, 6 M Urea, pH 6.5, mobile phase. The sample was diluted 1/20 in the mobile phase, and 50 μL were injected to the column. The flow rate was set at 0.5 ml/min, and elution off the column was monitored by a fluorescence detector set at an excitation wavelength of 295 nm, and emission wavelength of 360 nm (bandwidth 15 nm). The HPLC-SEC traces for the mPEG20k-Hz-BMP-7 conjugate, the oxidized BMP-7, and mature BMP-7 are shown in FIGS. 6A-6C, respectively.

For purification, the conjugate was dialyzed in 5 mM sodium phosphate buffer pH 7, using SPECTRA/POR 1 membrane tubing (Spectrum Medical Industries Inc., Los Angeles, Calif.), having a molecular weight cut-off of 6000-8000. The dialysis was carried out at 4° C. At the end of dialysis, the sample Was filtered through a 0.45 μm Acrodisc HT Tuffryn membrane syringe filter (PALL Life Sciences, Ann Arbor, Mich.).

Then, two sulphopropyl cation exchanger columns, HiTrap SP HP 5 ml each (GE Healthcare, Piscataway, N.J.), were connected in series to a total bed volume of 10 mL. The columns were equilibrated with 5 column volumes of 5 mM sodium phosphate buffer pH 7. Next, 25 mL of the dialyzed conjugation sample were loaded on the columns at a flow rate of 1 mL/min under 5 mM sodium phosphate pH 7. The unbound material to the column were collected and reloaded twice to the column under the same conditions described above. Three mobile phases were used in the elution process: mobile phase A containing 5 mM sodium phosphate buffer pH 7, mobile phase B1 containing 1 M NaCl in 20 mM sodium phosphate pH 7, and mobile phase B2 containing 6 M Urea, 1 M NaCl, 20 mM sodium phosphate pH 7. The elution started by increasing mobile phase B1 to 30% for 30 minutes at 2 mL/min, then to 100% B2 for 4 minutes at 4 mL/min. Fractions were collected at 5 mL/fraction throughout the entire separation. Unreacted PEG did not bind to the column, and came out with the flow-through fractions. However, PEGylated-BMP7 and unconjugated BMP7 were both eluted at 30% of mobile phase B1 (30 mM NaCl).

The fractions containing PEGylated-BMP7 and unconjugated BMP7 from the ion exchange chromatography separation were pooled and subjected to further purification by hydrophobic interaction chromatography in order to separate the PEGylated-BMP7 from the unconjugated BMP7. Three phenyl hydrophobic interaction columns, HiTrap phenyl Sepharose HP 5 ml each (GE Healthcare, Piscataway, N.J.), were connected in series and equilibrated with 100 mL of binding mobile phase A containing 1M ammonium sulfate, 6 M urea, and 20 mM sodium phosphate buffer pH 7. The pool sample was then injected to the columns at a flow rate of 1 mL/min. The flow through peak was collected in 5 mL fractions and re-injected in order to obtain maximum binding to the column. The gradient was then changed to 70% A and 30% B (6 M urea, 20 mM Na phosphate pH 7), and increased to 100% B over 70 minutes, followed by 20 minutes at 100% B. Fractions were collected at 1 mL/fraction throughout the elution stage. In order to identify the content of the fractions collected throughout the hydrophobic interaction chromatography separation, aliquots from the fractions were analyzed by HPLC-SEC using Superose6 10/300 GL column described above.

Fractions from the hydrophobic interaction separation containing the PEGylated protein were pooled concentrated and dialyzed in PBS pH 7.2, under nitrogen at 20 psi, in a 10 mL Amicon ultrafiltration stirred cell (Millipore Corp., Billerica, Mass.), using an OMEGA ultrafiltration membrane disc filter (PALL Life Sciences, Ann Arbor, Mich.), having a molecular weight cut-off of 3000. The sample volume was brought down to 4 mL final volume.

The concentrated sample was sterile filtered through 0.22 μm Acrodisc HT Tuffryn membrane syringe filter, and sterilely filled into autoclaved glass vials. All vials were stored at 4° C. Approximately, 3.4 mg of PEG20k-Hz-BMP-7 were obtained from the purification, as determined by the protein assay described in Example 1 above, which determined that the protein concentration in the PEG20k-Hz-BMP-7 sample was 0.95 mg/mL.

B. Characterization

The purified mPEG20k-Hz-BMP7 sample was analyzed by size exclusion chromatography using the Superose6 10/300 GL column described above. The sample was diluted to 1/10 in the mobile phase, and 50 μL were injected to the column. The flow rate was set to 0.5 mL/min, and elution off the column was monitored by a fluorescence detector set at an excitation wavelength of 295 nm, and emission wavelength of 360 nm (bandwidth 15 nm). Results are shown in FIG. 7.

### Example 3

### In Vitro Activity of PEG30k-BMP-7 Conjugate

The quantitation of activity is based on the induction of alkaline phosphatase specific activity in rat osteoblastic (ROS) cells. 30,000 cells/well (in 200 μL) were added to flat bottom plate and incubated overnight at 37° C. BMP-7 was diluted in acetate/mannitol buffer pH 4.5 to a concentration of 0.5 mg/mL. A 4 μg/mL working stock solutions of both PEG30k-BMP-7 (prepared as described in Example 1) and mature BMP-7 were made in acetate/mannitol buffer. Serial dilutions of both were made in F12 media with 2 mg/mL BSA.

50 μL of samples of the conjugate or the mature protein were added to the plate in triplicate. The final concentrations of the samples are as follows: 800, 400, 200, 100, 50, 25, 12.5, 6.25, 3.125 and 0 ng/mL. The plate was incubated for 40 to 56 hours.

Then, 150 uL of condition media was removed and discarded. 100 μL of a warmed 2% Triton-X 100 solution was added to each well. The plate was placed back into the incubator for 60 minutes to lyse the cells and release the alkaline phosphatase molecules. The plate was incubated at 4° C. overnight to assure completion of the extraction process.

Next, one 40 mg p-nitrophenylphosphate (PNPP) tablet was added to 40 mL of 0.1M glycine, pH 9.6, and the solution was warmed for 30 minutes in a water bath. The plate was removed from 4° C. and centrifuged for 10 minutes at 2600 rpm. 20 μL of supernatant from each well was transferred to a respective well of an empty Dynatech Immulon 2 plate. 100 μL of the PNPP solution was added to each well of the Immulon 2 plate, the plate was sealed, and incubated for 20 minutes. The reaction was stopped by adding 75 μL of 0.2N NaOH to each well. The plate was then read on a plate reader at 405 nm. The results are shown in FIG. 8.

### Example 4

### In Vitro Analysis of PEG30k-BMP-7 Coniugate

Mature BMP-7 was obtained and PEGylated BMP-7 conjugate was prepared as described in Example 1, with PEG molecular weight of 30,000 Daltons.

Human kidney (HK-2 cell line) cells were cultured in keratinocyte serum free medium supplemented with 5 ng/mL recombinant human epidermal growth factor (rHuEGF) and 0.05 mg/mL bovine pituitary extract (complete K-SFM) to ˜80% confluence in T25 or 150 mm dishes. Cells were rinsed with K-SFM without supplements and then treated for 6 hours with K-SFM (no supplements) alone or with the addition of 100 ng/mL BMP-7, 2 ng/mL TNF-α, or 100 ng/mL BMP-7 and 2 ng/mL TNF-α. RNA was isolated using Qiagen RNAeasy kit and RT-PCR was performed. IL-6 levels were attenuated in the presence of BMP-7.

The cells were cultured in complete K-SFM at 100,000 cells per well in a 24 well plate overnight. Medium was removed and replaced with K-SFM without supplements containing BMP-7 (0, 25, 50, 100, 200, 400 ng/mL), TGF-β1 (0, 0.31, 0.625, 1.25, 2.5 or 5 ng/ml) or TNF-α (2 ng/mL) plus BMP-7 or TGFβ1 for 24 hours. Supernatant were collected asceptically into sterile microfuge tubes, spun at 2000 rpm in a microcentrifuge to remove cell debris, and transferred to fresh tubes. Supernatants were stored frozen (−20° C.) until analysis by ELISA. IL-6 and IL-8 were quantitated by ELISA detection (R&D Systems) per manufacturers directions.

The results are shown in FIGS. 9A-9C and 10A-10C.

### Example 5

### In Vivo Evaluation of Injection Site after Injection of PEG30k-BMP-7 Conjugate

Mature BMP-7 was obtained and PEGylated BMP-7 conjugate was prepared as described in Example 1, with PEG molecular weight of 30,000 Daltons.

Twenty male rats were obtained and randomized into four groups for treatment. Each animal acted as its own control by delivering the mature protein or the PEGylated protein to the left flank and the control vehicle to the right flank. The treatment groups were as follows:

| Group No. | Compound | Delivery Route |
|---|---|---|
| 1 | mature BMP-7 | subcutaneous (s.c.) |
| 2 | PEG30k-BMP-7 | subcutaneous (s.c.) |
| 3 | mature BMP-7 | intramuscular (i.m.) |
| 4 | PEG30k-BMP-7 | intramuscular (i.m.) |

Injections of the protein at a dose of 100 μg/kg and in a mannitol acetate buffer carrier vehicle were given in the left flank. The right flank of each animal was injected with the mannitol acetate buffer vehicle by the same delivery route as the protein given to the right flank. A single injection was given and the animals were observed for two weeks post injection. No signs of obvious or palpable nodules formed at the injection site during this two week observation period.

Fifteen days after the injection, the rats were euthanized and x-rayed. There were no obvious bone nodules visible in the x-rays from all groups. However, upon dissection, bone nodules were found in the muscle of 4/5 animals in Group 3 treated with mature BMP-7 administered i.m. No bone nodules were observed in the animals treated with PEG30k-BMP-7 (i.m and s.c) or with control vehicle (Groups 2, 4).

Tissue samples were removed from the animals and placed into 5% PVA. They were frozen using a hexane freezing bath and placed into −80° C. freezer. The majority of these blocks were subsequently thawed in 70% alcohol and processed for microCT analysis. Bone was observed in 4/5 muscles in the animals receiving i.m. BMP-7 (Group 3) and 5/5 in the animals receiving s.c. BMP-7 (Group 1). The results are summarized in Table B.

TABLE B

| | | Dose | Dose | | | | |
|---|---|---|---|---|---|---|---|
| | | Level | Volume | Dose | Dose | | |
| Group | No. of | PEG- | PEG- | Level | Volume | Route of | Bone |
| Number | Animals | BMP7 | BMP7 | BMP7 | BMP7 | Administration | Formation |
| 1 | 5 | — | | 100 ug/kg | 100 μL | s.c. | 0/5 |
| 2 | 5 | 100 μg/kg | 100 μL | | | s.c. | 0/5 |
| 3 | 5 | — | | 100 ug/kg | 100 μL | i.m. | 4/5 |
| 4 | 5 | 100 μg/kg | 100 μL | | | i.m. | 0/5 |

### Example 6

### In Vivo Evaluation of Injection Site after I.M. Injection of PEG30k-BMP-7 Conjugate

Mature BMP-7 was obtained commercially and PEGylated BMP-7 conjugate was prepared as described in Example 1, with PEG molecular weight of 30,000 Daltons.

Ten male rats were obtained and randomized into two groups for treatment. Each animal acted as its own control by delivering the mature protein or the PEGylated protein to the left flank and the control vehicle to the right flank. The treatment groups were as follows:

| Group No. | Compound | Delivery Route |
|---|---|---|
| 1 | mature BMP-7 | intramuscular (i.m.) |
| 2 | PEG30k-BMP-7 | intramuscular (i.m.) |

Injections of the protein at a dose of 100 μg/kg and in a mannitol acetate buffer carrier vehicle were given in the left flank intramuscularly. The right flank of each animal was injected with the mannitol acetate buffer vehicle by the same delivery route as the protein given to the right flank. When giving the injection, the rats were anesthetized with a 70/30 mixture of CO₂/O₂. Care was taken to ensure that animal's bone was not hit by the needle or that bone was not too close to the injection site. A single injection was given and the animals were euthanized two weeks post injection for analysis.

X-ray analysis of the injection area showed bone nodules in all of the animals treated with mature BMP-7. None of the animals treated with PEG30k-BMP-7 (Group 2) developed bone nodules. At the time of dissection, nodules in the Group 1 animals treated with mature BMP-7 were palpable and upon dissection, bone nodules were visualized in the muscles of the animals in this group. Upon dissection of the animals in Group 2, no bone nodules were found. This data was confirmed by microCT analysis (4 out of 5 with bone). None of the vehicle injections placed into the right flank of each animal resulted in ossification. The results are summarized in Table C.

TABLE C

| | | Dose | Dose | | | | |
|---|---|---|---|---|---|---|---|
| | | Level | Volume | Dose | Dose | | |
| Group | No. of | PEG- | PEG- | Level | Volume | Route of | Bone |
| Number | Animals | BMP 7 | BMP7 | BMP 7 | BMP7 | Administration | formation |
| 1 | 5 | | | 100 μg/kg | 100 μL | i.m. | 5/5 |
| 2 | 5 | 100 μg/kg | 100 μL | | | i.m. | 0/5 |

### Example 7

### In Vivo Evaluation of Injection Site After Injection of PEG20k-Hz-BMP-7 Conjugate

Mature BMP-7 was obtained and PEGylated BMP-7 conjugate was prepared as described in Example 2, with PEG molecular weight of 20,000 Daltons.

Ten male rats were obtained and randomized into two groups for treatment. Each animal acted as its own control by delivering the mature protein or the PEGylated protein to the left flank and the control vehicle to the right flank. The treatment groups were as follows:

| Group No. | Compound | Delivery Route |
|---|---|---|
| 1 | mature BMP-7 | intramuscular (i.m.) |
| 2 | PEG20k-Hz-BMP-7 | intramuscular (i.m.) |

Injections of the protein at a dose of 100 μg/kg and in a mannitol acetate buffer carrier vehicle were given in the left flank intramuscularly. The right flank of each animal was injected with the mannitol acetate buffer vehicle by the same delivery route as the protein given to the right flank. A single injection was given and the animals were euthanized two weeks post injection for analysis.

X-ray analysis of the injection area showed no bone nodules in any animals, however dissection of the animals revealed bone nodules in the rats treated with mature BMP-7. Dissection showed no nodules present in any of the injection sites for the animals with PEGylated BMP-7. None of the vehicle injection sites resulted in ossification.

### Example 8

### Pharmacokinetics of PEG30k-BMP-7 Conjugate

Male Sprague-Dawley rats were injected with 250 μg/kg PEG30k-BMP-7, 250 μg/kg mature BMP-7, or saline. Serum was collected by orbital bleeding at time 0 and at 15 minutes, 30 minutes, 1 hour, 2 hours, 3 hours, and 6 hours post dosing. The concentration of BMP-7 in the serum was analyzed by ELISA according to the following protocol.

Plates (Nunc Immuno Plate II F96 MAXISORP) were coated with BMP-7 monoclonal antibody 1B12. A standard was prepared by diluting a BMP-7 reference standard in 50 mM Tris-buffered saline (TBS)+1% bovine serum albumin (BSA) to a concentration of 10 μg/mL. The standard was aliquoted and stored at −80° C.

A first dilution of 800 ng/mL of the 10 μg/mL reference standard in serum, plasma, or assay detergent diluent/sample buffer (50 mM TRIS, 150 mM NaCl, 0.1% SDS, 1% NP-40, 0.5% DOC pH 8.0) was prepared.

Then, a standard curve was determined by performing a series of 1:2 dilutions in serum, plasma, or assay detergent diluent/sample buffer. The standard concentrations (final) were: 800, 400, 200, 100, 50, 25, 12.5, 6.25, 3.125, 1.56, 0.78, 0.39, 0.195, 0.097, 0.048, and 0 ng/mL.

50 μL of the standard controls and of the test samples were added to the appropriate wells. 50 μL of assay detergent diluent/sample buffer was added to all wells. The plate was securely covered with a plate sealer and incubated for 2 hours at room temperature (20-25° C.) on a shaker (speed 4).

The plates were washed using BMP-7 wash buffer (1× borate buffered saline, 0.1% Tween 20, pH 8.0) for 2 cycles and the plates were tapped dry. A Creative BioMolecules Rabbit 5086 (BMP-7 polyclonal antibody) was diluted 1:2500 in BMP-7 conjugate diluent (50 mM TRIS, 150 mM NaCl, 1% BSA, pH 7.2) and 50 μL was added to all the wells. The plates were covered securely with a plate sealer and incubated for 1 hour at room temperature (20-25° C.) on a shaker. Then, the plates were washed on the plate washer, 2 cycles as described above. The plates were tapped dry and then 50 μL per well of a 1:25,000 dilution (in 1×PBS, 0.05% Tween-20, pH 7.4) of goat-anti-rabbit HRP conjugate (Zymed Cat # 81-6120) was added. The plates were again covered and incubated at room temperature (20-25° C.) on a shaker and then washed again.

100 μL per well of TMB Single Solution Substrate (Zymed Cat # 00-2023) was added for 5-10 minutes at room temperature. The reaction was stopped by adding 100 μL per well of stop solution (0.18M H₂SO₄). The absorbance was read at 450 nm. Results are shown in FIG. 11.

### Example 9

### In Vivo Efficacy of PEG30k-BMP-7 Conjugate

A unilateral ureteral obstruction (UUO) model was used to evaluate efficacy of the PEGylated PMB-7 conjugate. The ureter of mice was ligated to block ureteral flow. The ability of mature BMP-7 (SEQ ID NO:3), soluble BMP-7 (residues 30-431 of SEQ ID NO:1; mature domain with the amino terminal prodomain of the protein non-covalently in association with the mature BMP-7), and the PEG30k-BMP-7 conjugate (prepared as described above) to suppress the expression of markers of renal fibrosis, as a measure of ability to suppress progression of renal fibrosis was determined during a four day period of ureteral obstruction.

Mice were randomized into four treatment groups for treatment with vehicle alone (n=3), mature BMP-7 (n=4), soluble BMP-7 (n=4), or PEG30k-BMP-7 conjugate (n=4). All forms of BMP-7 were administered once per day at a concentration of 300 μg per kg of rodent weight.

The levels of beta actin, alpha smooth muscle, and collagen were determined from kidney tissue samples taken from each animal at the end of the four day period, using real time RT-PCR. The beta actin was measured by spiking a beta actin cDNA as a control. The level of expression in vehicle treated animals was set to one unit for convenient comparison. Results are shown in FIGS. 12A-12B.

### Example 10

### Immunogenicity of PEG30k-BMP-7 Conjugate

Male Balb/C mice are injected with PEG30k-BMP-7 or with mature BMP-7. Fourteen days later, the mice receive a second injection of the same test compound. Blood samples are drawn weekly from Day 0 to Day 35, serum is separated and the presence of anti-PEG-BMP-7 antibodies are detected using ELISA.

While a number of exemplary aspects and embodiments have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

It is claimed:

1. A composition comprised of an isolated bone morphogenetic protein covalently attached to a hydrophilic polymer.

2. The composition of claim 1, wherein said bone morphogenetic protein is bone morphogenetic protein-7.

3. The composition of claim 2, wherein said bone morphogenetic protein-7 is human recombinant bone morphogenetic protein-7.

4. The composition of claim 3, wherein said human bone morphogenetic protein-7 has a sequence identified herein as SEQ ID NO:3.

5. The composition of claim 1, wherein said hydrophilic polymer is poly(ethylene glycol).

6. The composition of claim 5, wherein said poly(ethylene glycol) has a molecular weight of between about 10-50 kDaltons.

7. The composition of claim 5, wherein said bone morphogenetic protein is conjugated via two or more amino acid residues to said hydrophilic polymer.

8. A pharmaceutical preparation comprised of the composition according to any preceding claim and a pharmaceutically-acceptable vehicle.

9. A method of treatment, comprising administering via injection the pharmaceutical preparation of claim 8.

10. A composition of conjugates comprised of bone morphogenetic protein-7 modified to include a covalently attached hydrophilic polymer, said composition of conjugates prepared according to the process of reacting said bone morphogenetic protein-7 with a functionalized hydrophilic polymer in the presence of an activating agent.

11. The composition of claim 10, wherein said activating agent is selected from the group consisting of N-hydroxysuccinimide (HOSu), 1-hydroxybenzotriazole (HOBt), and hydroxyl-7-azabenzotriazole (HOAt).

12. The composition of claim 10, wherein said functionalized hydrophilic polymer is an activated derivative of poly(ethylene glycol).

13. The composition of claim 12, wherein said activated derivative of poly(ethylene glycol) is nitrophenyl carbonate derivatized methoxy-polyethylene glycol.

14. The composition of claim 10, wherein said bone morphogenetic protein-7 is human bone morphogenetic protein-7.

15. The composition of claim 14, wherein said conjugates form a heterogenous composition of conjugates having a ratio of PEG:bone morphogenetic protein-7 of 1:1, 2:1 and 3:1.

16. The composition of claim 14, wherein said composition comprises more than about 50% of conjugates having a 1:1 ratio of PEG:bone morphogenetic protein-7.


SEQUENCE LISTING (3 sequences)

SEQ ID NO: 1 - protein, 430 aa, Homo sapiens
MHVRSLRAAAPHSFVALWAPLFLLRSALADFSLDNEVHSSFIHRRLRSQERREMQREILSLGLPHRPRPHLQGKHNSAPMFMLDLYNAMAVEEGGGPGGQGFSYPYKAVFSTQGPPLASLQDSHFLTDADMVMSFVNLVEHDKEFFHPRYHHREFRFDLSKIPEGEAVTAAEFRIYKDYIRERFDNETFRISVYQVLQEHLGRESDLFLLDSRTLWASEEGWLVFDITATSNHWVVNPRHNLGLQLSVETLDGQSINPKLAGLIGRHGPQNKQPFMVAFFKATEVHFRSIRSTGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH

SEQ ID NO: 2 - protein, 263 aa, Homo sapiens
ADFSLDNEVHSSFIHRRLRSQERREMQREILSLGLPHRPRPHLQGKHNSAPMFMLDLYNAMAVEEGGGPGGQGFSYPYKAVFSTQGPPLASLQDSHFLTDADMVMSFVNLVEHDKEFFHPRYHHREFRFDLSKIPEGEAVTAAEFRIYKDYIRERFDNETFRISVYQVLQEHLGRESDLFLLDSRTLWASEEGWLVFDITATSNHWVVNPRHNLGLQLSVETLDGQSINPKLAGLIGRHGPQNKQPFMVAFFKATEVHFRSIR

SEQ ID NO: 3 - protein, 139 aa, Homo sapiens
STGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNMVVRACGCH